(19) United States
(12) Patent Application Publication
(10) Pub. No.: US 2005/0143684 A1
(43) Pub. Date: Jun. 30, 2005

(54) Apheresis methods and devices

(76) Inventors: Charles Bolan, Silver Spring, MD (US); Susan F. Leitman, Potomac, MD (US); Herb Cullis, Gaithersburg, MD (US)

Correspondence Address: Attn: John J. Gresens;MERCHANT & GOULD P.C., P.O. Box 2903, Minneapolis, MN, 55402-0903, US

(21) Appl. No.: 11/067,405

(22) Filed: Feb. 22, 2005

Related U.S. Application Data
(63) Continuation of application No. 10/008,857, filed on Nov. 2, 2001.
(60) Provisional application No. 60/245,901, filed on Nov. 3, 2000.

(51) Int. Cl. A61M 37/00

(52) U.S. Cl. 604/6.01; 604/6.16

(57) ABSTRACT

An apheresis method that includes drawing blood from a mammal, adding an amount of an agent effective in preventing coagulation, wherein the agent is an anticoagulant, extracting one or more constituent components from the blood, wherein an extracted blood and constituent component result therefrom, and diminishing the activity of said anticoagulant by introducing an antidote, wherein the amount of antidote introduced is coupled with the amount of anticoagulant added. The antidote is provided either to the processed blood prior to reintroduction to the donor or directly to the donor. The invention also includes an apheresis machine that includes an antidote delivery conduit, wherein the antidote delivery conduit delivers an amount of antidote that is coupled with an amount of anticoagulant delivered.

This application claims priority to U.S. Provisional App. No. 60/245,901, filed Nov. 3, 2000 entitled APHERESIS METHODS AND DEVICES.

### FIELD OF THE INVENTION

This invention relates generally to the field of apheresis of a particular constituent component of blood such as platelets, leukocytes, erythrocytes, or plasma, from a donor or a patient in a process where blood is withdrawn, anticoagulated, and the desired constituent is isolated and collected while the extracted blood and anticoagulant are reinfused to the donor.

### BACKGROUND OF THE INVENTION

In the practice of medicine, constituent components of blood are donated by one individual donor for transfusion into another individual patient for the purposes of improving the health of the other individual. The most common process is that of donating red blood cells through collection of whole blood for transfusion to patients who are deficient in red blood cells. The process by which stem cells or platelets are donated is referred to as apheresis. Any particular blood component can be removed from whole blood; platelets, leukocytes, erythrocytes, or plasma.

During apheresis, the whole blood removed from the donor must be anticoagulated to prevent clotting in the apheresis device, the desired component is then isolated and collected, and the remainder of the blood is reinfused to the donor along with the anticoagulant. At times, apheresis therapy is used therapeutically for a patient who is ill rather than for a healthy donor, in which case apheresis removes an undesired component of the blood, which may be contributing to medical illness, replacing it with components that may improve health.

Systems for apheresis are well known in the art such as those disclosed in U.S. Pat. No. 3,655,123 (Judson et al.), U.S. Pat. No. 4,120,448 (Cullis), U.S. Pat. No. 4,146,172 (Cullis et al.), U.S. Pat. No. 4,185,629 (Cullis et al.), U.S. Pat. No. 4,187,979 (Cullis et al.), U.S. Pat. No. 4,540,397 (Lolachi et al.), U.S. Pat. No. 4,850,995 (Tie et al.).

Citrate solutions have been utilized to prevent blood coagulation in transfusion medicine procedures for more than eight decades, and are the anticoagulant of choice in apheresis. The safety and tolerability of infused citrate may be due to several factors including the physiologic presence of small quantities of citrate in blood, large stores of citrate in bone, and the integral role of citrate in cellular and mitochondrial metabolism.

Citrate is normally present in blood at a concentration of approximately 0.1 to 0.2 mmole/L blood. In apheresis processes citrate is often administered when incorporated into various solutions containing dextrose, pH adjusting acids, phosphate, adenine, and buffering agents, the most common being acid citrate dextrose or ACD-A. Other anticoagulants include EDTA (ethyldiaminetetraaceticacid) and heparin. EDTA and heparin are not used as extensively as ACD-A because they produce side effects that are often considered unacceptable in healthy volunteer donors and undesirable in patients. During administration of commercial anticoagulant solutions containing trisodium citrate and citric acid, coagulation is inhibited in the apheresis device and product by both decreased ionized calcium levels and decreased pH. High concentrations of citrate lead to anticoagulation by binding to, combining with, and forming soluble complexes with calcium ions, which are necessary for coagulation. These soluble citrate complexes are not available for further chemical reaction. When the concentration of ionized calcium falls below 0.3 mmole/L, the process of clot formation is prevented, and anticoagulation has occurred.

The ratio of whole blood to citrate anticoagulant (which determines the concentration of citrate in the blood) is thus a critical determinant of smooth, uneventful processing of blood during apheresis and of product stability. When increasing ratios of whole blood to citrate are utilized, product clumping and blood coagulation may occur because the concentration of citrate in the apheresis device and product is decreased. Therefore, apheresis procedures generally require a minimum whole blood to citrate anticoagulant ratio.

During the process of apheresis, anticoagulant is returned to the donor along with the returned blood. The rate of anticoagulant delivery is determined by the whole blood to anticoagulant ratio utilized and the average whole blood flow rate. The rate of whole blood flow is determined by the rate of blood processing in the apheresis device minus the amount of blood collected in the product. Generally this results in an average of about 1.0 to 2.2 mg citrate/kg body weight/minute.

In continuous apheresis procedures, the blood processing and return of blood to the donor are done concurrently. In discontinuous apheresis procedures the blood processing and return of blood are performed at distinct time intervals. The return of the processed blood is accomplished by a rapid bolus with high citrate infusion rates undertaken for a short period of time. Over the entire course of apheresis, the rate of return of citrate is generally similar in discontinuous and continuous procedures.

This citrate administration protects blood from coagulation in the apheresis device. However, citrate administration results in symptoms when returned to the donor because ionized calcium levels decrease to a degree that does not significantly inhibit coagulation but which produces neuromuscular complications. This is because blood citrate levels are not as high in the donor circulation as they are in the apheresis device due to redistribution and metabolism of citrate in the donor circulation after return of the citrated blood during apheresis. Upon reinfusion of the citrate blood into the donor, the human body also mobilizes calcium from bone and other reserves to counteract the excess citrate and restore or maintain free calcium concentrations. The body excretes the calcium citrate precipitate complexes in the urine. In addition to calcium, other positively charged molecules, such as magnesium, may also be complexed by citrate, resulting in decreased ionized levels in the same fashion as ionized calcium levels are decreased. This can produce additional complications in the donor.

In most donors, the human body mobilizes calcium, without any detrimental effect from the apheresis procedure, as long as citrate levels do not rise to levels that produce rapid or prolonged decreases in ionized calcium. However, if the body is unable to mobilize enough calcium to restore ionized calcium levels in the blood, as the citrate levels increase, symptoms associated with the decreased ionized calcium level will manifest themselves. Some donors experience transient hypocalcemia symptoms such as a feeling of numbness, coldness, or tingling in the extremities, mouth, or chest. With a continued rise in the citrate levels, these symptoms become extremely uncomfortable and may result in cardiovascular collapse and death. The rate at which apheresis can be conducted is thus limited in all donations by the rate of return of citrate to the donor, and many lighter weight donors cannot tolerate blood processing rates associated with economically feasible apheresis procedures.

In contrast to platelet collections (which usually last one to two hours and process 5 liters of donor blood), large volume leukapheresis (LVL), lasts several hours and may process 10-25 liters of whole blood repeatedly over several days to obtain doses of hematopoietic progenitor cells and mononuclear cells for modern transplantation and other complex therapies. Citrate infusion rates utilized in LVL are generally extrapolated from much shorter plateletpheresis procedures, which usually process 5 liters of whole blood over 90 to 120 minutes.

Donor responses during common citrate infusion rates for LVL remain incompletely characterized, and the rate of returned citrate and associated citrate-related donor symptoms are a major limitation to the rate of blood processing. This results in longer procedures being necessary to obtain the desired product content. Thus a single 15 L procedure can require a processing time ranging from about 5 to 9 hours for a 45 kg donor, and from about 3 to 5 hours for an 80 kg donor when LVL is performed at standard citrate infusion rates and whole blood to citrate anticoagulant ratios. Even when using the citrate infusion rates used in plateletpheresis, the LVL donor may have symptoms because the procedure lasts longer than the plateletpheresis procedure and citrate levels continue to rise throughout. This may become even more significant when the stem cell donor has a low concentration of stem cells in the blood despite medications that may be given to increase the stem cell count. This situation necessitates that the procedure be performed for even longer periods of time, and therefore results in greater accumulation of citrate. Reducing citrate related symptoms while still increasing citrate infusion rates in LVL would be of great benefit to stem cell donors, and to platelet donors in whom the duration of the platelet procedure and the dose obtained are limited by the rate of processing the blood due to the need to prevent the return of citrate to the donor from reaching toxic levels.

During therapeutic apheresis procedures, the apheresis device may serve to remove an undesired blood component such as excessively high platelets, white blood cells, or plasma containing toxins or inhibitors. Alternatively apheresis processes may be used to add large volumes of a blood component which is deficient. Therapeutic procedures also utilize citrate anticoagulant to prevent coagulation in the apheresis device.

In addition, these procedures may introduce additional amounts of citrate contained in the red blood cells or plasma which may be added as a replacement fluid. Some examples include removal of platelets for excessive thrombocytosis, removal of white blood cells for hyperleukocytosis, removal of plasma for myasthenia gravis, and removal of plasma and replacement of plasma for thrombotic thrombocytopenia purpura (TTP). The total amount of citrate returned to the donor is thus a function of the portion of the citrate anticoagulant used in the apheresis which is returned to the donor and the citrate contained in the blood or other components which are therapeutically added. This total amount of returned citrate causes symptoms in the donor in the same manner as the citrate administered during plateletpheresis or LVL as described above.

In addition to the ionized calcium depletion that occurs when blood with excess citrate is reinfused to the donor, the decrease in blood ionized calcium levels in blood triggers a metabolic response in the donor to elevate the parathyroid hormone (PTH) levels of the donor. This and other processes trigger calcium mobilization. This mobilization occurs within six minutes of citrate administration during blood reinfusion and prepares the body for the rapid assimilation of calcium. However, this process cannot counteract the infusions of citrate required to obtain desired stem cell and platelet doses.

The state of the art in medicine is to accept these changes in ionized calcium concentration as a required part of the process. However, some studies have recommended injection of calcium into the donor to alleviate the associated symptoms. This approach is not widely practiced because many apheresis practitioners consider the use of calcium injections as unsafe despite the large decrease in ionized calcium that occur in apheresis. Calcium depletion in the donor during apheresis has also been monitored by rapid laboratory analysis, and a calcium replacement amount has been calculated and injected thereby. Although these studies have begun to offer a solution to the problem, no efficient, inexpensive and easily automatable solution currently exists.

As a result, there is a need for efficient, safe and symptom free apheresis processes and devices that address the problem of the ion depletion which occurs due to the return to the donor of the citrate anticoagulant necessary to prevent coagulation in the apheresis device.

### SUMMARY OF THE INVENTION

The invention is a device and method useful in apheresis procedures in mammals. The invention offers a method of conducting apheresis comprising the steps of drawing mammalian blood into an apheresis device, adding a measured amount of agent effective in preventing coagulation wherein the agent comprises an anticoagulant which is added to the mammalian blood soon after withdrawal of the blood to prevent coagulation in the apheresis device, extracting one or more constituent components from said mammalian blood, and when the blood is returned to the mammalian circulation, diminishing the activity in the mammalian circulation of said agent used in preventing coagulation in the apheresis device, wherein the activity of said anticoagulant is diminished by the introduction of an ionic agent at a concentration determined by the concentration or calculated, expected concentration of said anticoagulant, with the introduction of said ionic agent used to diminish the coagulation being accomplished after the blood is processed in the apheresis device at a point prior to returning to the circulation.

In this manner, anticoagulation is achieved in the apheresis device, and a measured amount of an agent that counteracts the harmful effects produced in the donor or patient by the anticoagulant is added to the blood just prior to returning to the donor after the blood has been separated in the apheresis device.

Preferably, the apheresis procedures of the invention are carried out on mammals such as pigs, primates, canines, and humans. More preferably, the apheresis procedures of the invention are carried out on humans. Preferably, the agent effective in preventing coagulation comprises, a citrate compound, heparin, EDTA, or combinations thereof. More preferably, the agent effective in preventing coagulation comprises a citrate compound, such as acid citrate dextrose (ACD-A). Preferably, the ionic agent utilized to diminish the activity of the anticoagulant is a solution or combination of solutions comprising calcium, magnesium or a combination thereof, which may include other substances diminished by the citrate anticoagulant such as potassium. More preferably, the ionic agent utilized to diminish the activity of the anticoagulant is a solution comprising calcium chloride, calcium gluconate, magnesium sulfate, salts of these compounds, or other desired electrolytes, or combinations thereof.

The invention also offers a device capable of carrying out the method of the invention. The device is an apheresis machine wherein the anticoagulant solution is coupled to a solution of the ionic agent. Preferably, the anticoagulant solution is coupled to the solution of the ionic agent by electrical, mechanical or hydraulic means; or by correlating the concentrations of the anticoagulant and ionic agents. More preferably, the anticoagulant solution is coupled to the solution of the ionic agent by electrical or mechanical means. The amount of anticoagulant delivered during a time period is coupled to the amount of ionic agent delivered during a time period so that a measured dose of the ionic agent is administered to the patient.

Preferably, this coupling of the anticoagulant solution to the ionic agent solution is accomplished by utilizing the same pump for delivery of the two solutions, or connecting the two separate pumps electrically to deliver the two solutions. The coupling may be direct based on the actual flow of the anticoagulant solution, or it may be indirect based on the anticipated delivery of anticoagulant during the apheresis procedure. The apheresis machine of the invention can either be constructed by modifying existing apheresis machines or by constructing entirely new machines. Thus a measured amount of a compound is added with the return of blood to the mammalian circulation that alleviates toxic effects of the anticoagulant solution which is used to prevent coagulation in the apheresis device or collected product, whether these toxic effects be due to neuromuscular effects from low cation levels, depletion of other electrolytes, or other effects, and that the amount of this compound is based on the amount of anticoagulant used during the apheresis procedure, and that the device automatically links administration of the alleviating compound with the amount of anticoagulant that is used. The measured amount of anticoagulant administered can be modified according to the medical condition of the donor. For example, liver or kidney disease may alter the metabolism of the returned citrate. Also, citrate levels may be modified by the presence of mild symptoms in the donor, resulting in increasing the measured amount of calcium or other substances delivered in relationship to the administration of citrate.

### BRIEF DESCRIPTION OF THE DRAWINGS

FIG. 1 depicts a schematic representation of a standard dual arm apheresis system.

FIG. 2 depicts a schematic representation of a standard single arm apheresis system.

FIG. 3 depicts a schematic representation of one embodiment of the invention that utilizes a dual arm apheresis system.

FIG. 4 depicts a schematic representation of another embodiment of the invention that utilizes a dual arm apheresis system.

FIG. 5 depicts a schematic representation of one embodiment of the invention that utilizes a single arm apheresis system.

### DETAILED DESCRIPTION OF THE INVENTION

This invention includes methods of conducting apheresis and a device useful in apheresis techniques.

Blood is a circulating connective tissue comprising plasma, erythrocytes or red blood cells, leukocytes or white blood cells, and platelets. Whole blood is blood that has not been separated into its various constituent components. Constituent components of blood include plasma, erythrocytes, leukocytes and platelets. Mammalian blood is blood that is found in a mammal. Mammals are generally animals that (1) have hair, (2) provide milk for their young from specialized glands (mammary glands), and (3) maintain a high body temperature by generating heat metabolically. Exemplary mammals include rats, mice, pigs, primates, canines, cows, cats, and humans.

Apheresis is a technique in which blood is drawn from a donor and the desired constituent components are extracted and collected. The rest of the blood is returned to the donor. A donor can be any mammal. Apheresis can either be continuous, as in a dual arm procedure, or discontinuous, as in a single arm procedure. In continuous apheresis, blood is withdrawn, processed, and reinfused simultaneously in a continuous fashion. In discontinuous apheresis, blood is withdrawn, processed in small, discrete volumes while no reinfusion of blood is taking place and then blood withdrawal is discontinued while reinfusion is accomplished. In the context of apheresis procedures, withdrawn blood can either refer to an amount of blood that is drawn as one discrete volume of a discontinuous procedure, an amount of blood that is withdrawn as a result of the total procedure, or any amount in between.

Apheresis may also be used to conduct therapeutic plasma exchange processing (TPE). TPE is a therapeutic procedure in which a machine is used to extract plasma from a patient's blood, replace the plasma component with another fluid (including but not limited to fresh frozen plasma, plasma protein fractions, albumin preparations, dextran solutions or saline), and return the modified blood to the patient. In TPE, an anticoagulant is initially added to the withdrawn blood in order to aid in plasma extraction. This anticoagulant generally is removed with the plasma. However, before the blood is returned to the patient, more anticoagulant is added to the fluid returned to the patient.

During apheresis, the drawn blood may also be subjected to dialysis. Dialysis is a procedure in which a machine is used to filter waste products from the blood of a patient. Once the waste products have been removed from the patient's blood, it is returned to the patient. During dialysis, anticoagulants are generally added to the blood to aid in the filtration and processing. The most common anticoagulant is heparin, however citrate may be used in dialysis patients when there is a low platelet count or some other condition which may cause bleeding. In this case, the patient may be susceptible to citrate toxicity.

Blood clotting or coagulation is a phenomenon that requires platelets and at least fifteen factors normally present in blood or on cell membranes. Clotting results in blood losing its fluid liquid state and becomes clumped or coagulated. Blood clotting occurs through a sequence of events that culminates in a cascade of chemical reactions that cause the formation of an insoluble network of fibrin molecules that enmesh erythrocytes and platelets to form a blood clot. Clotting of compounds obtained from blood such as plasma, platelets, or red cells occurs by the same process.

Anticoagulants are normally administered to prevent blood clotting or coagulation. Anticoagulants function by modifying a necessary step or component in the sequence of events that cause blood clotting. Exemplary anticoagulants include but are not limited to heparin, warfarin, dicumarol, EDTA, oxalate, fluoride, and citrate solutions. Citrate solutions include any solution that has citrate ions or citric acid. Examples of citrate solutions useful as anticoagulants include but are not limited to trisodium citrate, acid citric dextrose (ACD-A), citrate phosphate dextrose (CPD), and citrate phosphate dextrose adenine (CPDA). Acid citrate dextrose (ACD-A) solutions are well known in the art and are commercially available. For example, an ACD-A solution containing dextrose and 21 mg/ml of citrate as citric acid and trisodium citrate can be commercially obtained from Baxter Healthcare (Fenwal Division, Baxter, Deerfield, Ill.).

Citrate anticoagulants in particular function by complexing with calcium and lowering available ionized calcium levels to a level such that coagulation does not occur. Citrate compounds also produce complexes with calcium, magnesium and other ions to a level that does not prevent coagulation, but which can cause neuromuscular irritability and other symptoms in mammals. Citrate compounds are also metabolized, resulting in changes in pH, bicarbonate and potassium when the citrate is returned to the donor. Also, the decreased levels of ionized calcium, though not sufficient to prevent coagulation in the mammalian blood, can result in release of hormones such as parathyroid hormone.

In methods of the invention, the activity of the agent effective to prevent coagulation is diminished by the introduction of an antidote. In one embodiment of the invention, the antidote comprises an ionic agent. Preferably, the ionic agent comprises a cation. More preferably, the ionic agent comprises a solution comprising calcium, magnesium, potassium or combinations thereof. Most preferably, the ionic agents of the invention comprise solutions comprising calcium chloride and magnesium sulfate. In another embodiment of the invention, the antidote comprises protamine. Protamine may be used to counteract the anticoagulating activity of heparin.

An ionic agent is a solution comprising an ion, examples of which are calcium, magnesium, potassium ions or combinations thereof. An ionic agent can comprise solutions of calcium chloride, calcium gluconate, magnesium sulfate, potassium chloride or combinations thereof.

Coupling may be used to correlate the amount of the anticoagulant delivered to the amount of the antidote delivered. The coupling of one agent to another generally means that the delivered amount of one agent is dependent, at least in part, on the delivered amount of another agent. Coupling can be done by mechanical, hydraulic, or electrical means. Coupling can also be accomplished by correlating the concentration of the two agents together so that the delivered amount of one is related to the delivered amount of the other agent. Generally, two agents are coupled together in an ongoing or continuous manner. Two agents may also be coupled if the agents are delivered at discrete times. For example, in a discontinuous process, the processed blood is returned when blood is not being withdrawn. In this example, the anticoagulant is still coupled to the antidote because the antidote delivery rate is based on the rate at which anticoagulant was initially added.

Similarly, two agents are not coupled if delivery of one agent is stopped, started, or adjusted without regard to the delivery of the other agent. However, two agents can be coupled while at the same time modifying the delivery amount of one based on patient symptoms or laboratory results.

### Methods of the Invention

Exemplary methods of coupling the agents include using the same pump for the two agents, electrically connecting two different pumps delivering the two agents, hydraulically connecting the two pumps or agents, or simply using two separate pumps at the same or different rates and coupling by preparing the concentrations of the two agents at specified concentrations.

Methods of the invention can also deliver the antidote and processed blood to the patient at different times or in different manners. For example, the antidote can be added to the extracted blood and then the mixture of the two can be returned via one line to the patient. Alternatively, the extracted blood can be returned, the antidote added to the extracted blood line, and then the mixture returned to the patient. Yet another alternative is to return the extracted blood via one line, and the antidote via another line simultaneously.

Methods of the invention comprise the steps of drawing blood from a mammal, adding an amount of an agent effective in preventing coagulation wherein the agent comprises an anticoagulant, extracting one or more constituent components from said blood, and diminishing the activity of said anticoagulant through introduction of an antidote, wherein the activity of said anticoagulant is diminished by the introduction of an antidote wherein the introduced amount is coupled to the introduced amount of said anticoagulant. In an alternative embodiment, the antidote is added to the blood just prior to the reinfusion of the blood into the mammal, after the anticoagulant has performed the necessary function of preventing coagulation in the apheresis device or in the collected product.

Any anticoagulant known to those of skill in the art may be used in the processes of the invention. The amount of the agent effective to prevent coagulation is well known to those of skill in the art, and standard texts and methods discussing apheresis procedures can be consulted for this information Apheresis Principles and Practice. Bruce C McCleod editor. AABB Press, Bethesda Md. 1997. Preferably, the anticoagulant comprises citrate compounds, heparin, or EDTA, or combinations thereof. More preferably, the anticoagulant comprises acid citrate dextrose (ACD-A). If the anticoagulant is ACD-A, an amount effective to prevent coagulation ranges from about 30 parts whole blood to one part ACD-A to 8 parts whole blood to one part ACD-A or other ranges depending on the desired product and procedure. The specific amount depends in part on the type of apheresis procedure being carried out. If the anticoagulant is not ACD-A the amount of citrate to be delivered to the whole blood can be determined by one of skill in the art and can be contained in the anticoagulant.

The constituent components extracted from the drawn blood comprises any one or more of the following: plasma, leukocytes, erythrocytes, or platelets. In one embodiment of the invention, the constituent components extracted from the drawn mammalian blood preferably comprise leukocytes. One preferred use of the method of the invention is in large volume leukapharesis (LVL). LVL procedures are employed to obtain allogenic peripheral blood stem cells (PBSC) for hematopoietic transplantations and other cellular therapies. LVL procedures generally process 10-25 L of donor blood daily over several days to obtain the desired amounts of leukocytes for hematopoietic stem cell transplantation or other procedures involving complex post-harvesting processing. The method of the invention is very beneficial to LVL procedures, as it allows the procedure to be done in less time and with less discomfort to the donor.

Apheresis procedures may also be used to collect plasma (plasmapheresis) to be used for therapeutic or commercial purposes. In these procedures, most of the citrate anticoagulant is removed with the collected plasma, however a significant portion may still remain with the blood returned to the donor. Despite the reduced amount of citrate that is returned to the donor, in plasmapheresis performed at high processing rates in commercial settings, significant citrate toxicity may still limit the rate of collection. Thus adjustment of the rate of administration of an ionic agent, such as calcium, based on donor symptoms or the expected rate of return of anticoagulant could also allow faster plasmapheresis procedures.

In yet another embodiment of the invention, the constituent components extracted from the blood, preferably comprise plasma. A most preferred use of methods of invention is therapeutic plasma exchange (TPE). TPE procedures are employed to extract plasma from a patient's blood and replace the plasma component with another fluid. TPE procedures are accomplished by drawing blood, adding an anticoagulant, extracting plasma, resulting in extracted blood and plasma, adding further anticoagulant to the extracted blood, diminishing the activity of the later added anticoagulant , an antidote, and reinfusing the extracted blood to the patient.

In one type of TPE procedure, the replacement fluid is albumin, which contains no citrate anticoagulant. This albumin replaces the plasma as well as the albumin withdrawn from the donor. Because albumin binds to calcium, calcium balance may be disrupted and net calcium loss from the body may occur.

In another type of TPE procedure, the replacement fluid is fresh frozen plasma (FFP) containing fibrinogen, clotting factors and other proteins. During the initial collection of FFP prior to storage, citrate is added to prevent coagulation due to the contained clotting factors. When infused to the donor as a replacement fluid, this additional citrate is also infused into the donor. At the same time, most of the donor's citrated plasma is removed from the donor, while citrate contained in the donors red blood cells is returned to the donor. TPE procedures using albumin replacement typically process 1 to 2 donor plasma volumes every 2 to 4 weeks, but sometimes more frequently. TPE procedures using FFP for treatment of TTP may process as much as 2.5 donor plasma volumes daily for several weeks at a time.

Dialysis refers to procedures in which toxic molecules are removed from the body by flowing blood over semi-permeable membranes through which the molecules pass into a dialysate fluid, often in patients with kidney diseases. Anticoagulation also must be used during dialysis to prevent blood clotting. Because the flow of blood is much higher than during apheresis (in order to efficiently remove the toxic waste molecules) heparin is usually used as an anticoagulant. When citrate is used for patients who may be at risk from bleeding due to heparin, citrate toxicity is common due to the high flow rates. Such citrate toxicity may be counteracted using calcium and other ionic solutions as described above.

In one embodiment of the invention, the introduction of the ionic agent may also be effective at replacing electrolytes removed from the blood during the extraction of the constituent components or by the action of the anticoagulant. The ionic agent can be effective in this capacity by inclusion of the calcium or magnesium, as well as other ions that may be removed in the process of apheresis. Methods of the invention include the addition of other compounds, and other ions, either cations or anions, to the ionic agent to increase the concentration of various electrolytes that may have been removed in the apheresis process. For example, potassium ions could be added to the ionic agent.

The amount of the antidote introduced is coupled to the amount of the anticoagulant introduced. The concentration of the antidote, as well as the rate at which the antidote is introduced can be modified to render the antidote capable of diminishing the activity of the anticoagulant. The amount of the antidote introduced can also be defined as a ratio of the amount of the anticoagulant that was initially added. The amount of the antidote introduced can also be defined based on a known amount of anticoagulant that will be given during an apheresis procedure of a given duration and/or amount of blood processing.

In one embodiment of the invention, the amount of antidote introduced can be related to the amount of the anticoagulant agent. For example, the antidote can be introduced at a particular amount of cation per volume of specified concentration and identity of anticoagulant solution. For example, the introduction of about 0.5 mg calcium ion per ml of ACD-A (solution containing dextrose and 21.4 mg/ml of citrate as citric acid and trisodium citrate (Baxter Healthcare, Fenwal Division, Deerfield, Ill.) may be effective in preventing hypocalcemic symptoms in the donor. The antidote is preferably introduced from about 0.25-1.5 mg calcium ion per ml of ACD-A. Preferably the ionic agent is introduced from about 0.5-1 mg calcium ion per ml of ACD-A. More preferably, from about 0.5-0.7 mg calcium ion per ml of ACD-A. Alternatively, the amount of antidote introduced can be varied in concert, or separate from the delivery of the anticoagulant. Also, in accordance with the invention, the antidote and anticoagulant are introduced substantially simultaneously in accordance with the manner in which these agents are to be coupled, e.g., metered by concentration. The amount of ionic agent may be further adjusted based on donor symptoms, or laboratory measurements obtained for example in automated or manual mode.

The quantity of antidote introduced can also be related to the quantity of anticoagulant introduced. For example, the antidote can be introduced at a number of mmoles of cation per number of mmoles of anticoagulant molecule. For example, the introduction of about 1 mmoles calcium to 10 mmoles citrate could be effective in preventing significant donor symptoms. Methods of the invention comprise administering from about 0.1-10 mmole calcium per 10 mmoles citrate (about 0.01-1 mmol calcium per 1 mmol citrate). Preferably, methods of the invention comprise administering from about 0.1-2 mmole calcium per 10 mmoles citrate (about 0.01-0.2 mmoles calcium per 1 mmol citrate). More preferably, methods of the invention comprise administering from about 1-1.3 mmole calcium per 10 mmoles citrate (about 0.1-0.13 mmoles calcium per 1 mmol citrate).

Methods of the invention further comprise administering from about 0.15-5 mmoles magnesium per 10 mmoles citrate. Preferably, methods of the invention comprise administering from about 0.5-1 mmoles magnesium per 10 mmoles citrate. More preferably, methods of the invention comprise administering about 0.5-0.6 mmoles magnesium per 10 mmoles citrate.

The magnesium administered can also be measured based on the volume of anticoagulant introduced, and in that case, the method comprises administering from about 0.1-0.5 mg magnesium/ml ACD-A. Preferably, methods of the invention comprise administering from about 0.15-0.3 mg magnesium/ml ACD-A. More preferably methods of the invention comprise administering about 0.15 mg magnesium/ml ACD-A.

Methods of the invention also comprise administration of other electrolytes that may have been removed from the blood during a procedure such as apheresis or dialysis. The amount of the electrolyte administered to the donor would be correlated to the rate of anticoagulant introduction or blood processing rate. Alternatively, the amount of an electrolyte administered may also depend in part on the duration of the procedure and the number of procedures that have been performed.

### Devices of the Invention

Machines such as those disclosed in the patents listed above can be modified and improved upon in the present invention. In order to understand devices and methods of the invention, the functioning of standard apheresis systems, such as those discussed above, will first be explained.

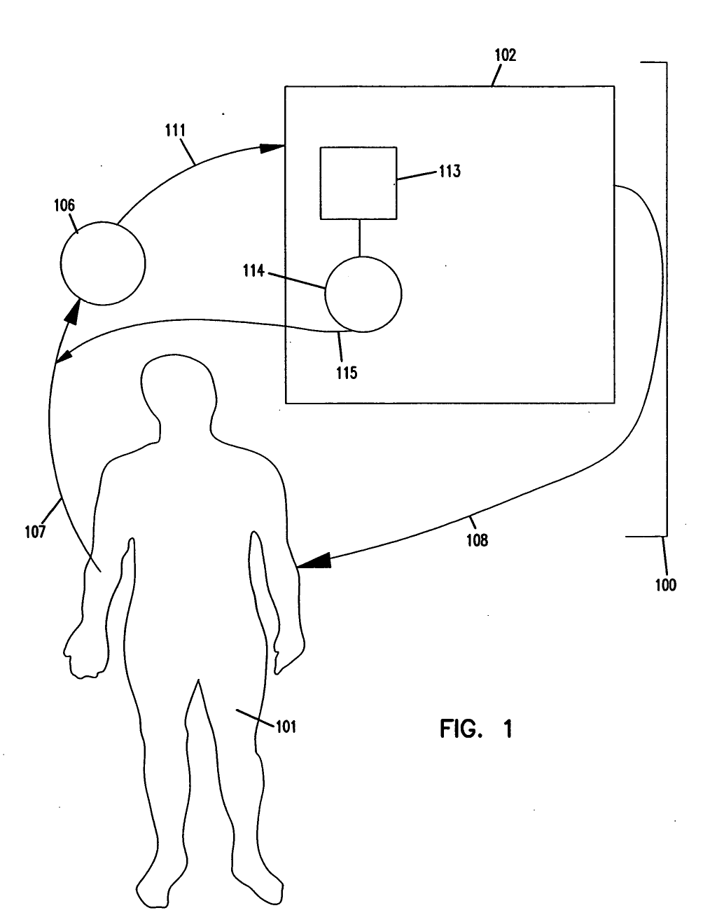

A general schematic of the functioning of one type of apheresis system, a dual arm system, is depicted in FIG. 1. The dual arm apheresis system 100 can be chosen from any of the currently available types of dual arm apheresis systems, examples of which are Baxter-Fenwal CS-3000 Cell Separator or Baxter-Fenwal Amicus (Baxter, Deerfield Ill.) or Cobe Spectra (Cobe BCT, Lakewood Colo.) or Fresenius AS-104 (Fresenius USA, Walnut Creek Calif.), or any other dual arm apheresis systems developed in the future. As is standard in apheresis systems, the blood withdrawal conduit 107 removes blood from the patient 101, through use of a first pump 106. The first pump 106, as well as all other pumps in apheresis systems and devices of the invention, can be peristaltic, piston, pneumatic, hydraulic pumps, or other pumps known to those of skill in the art, or disclosed in the previously referenced patents.

An anticoagulant, contained in a first compartment 113, is delivered to the withdrawal conduit 107 by an anticoagulant pump 114 via an anticoagulant delivery conduit 115.

The whole blood extracted from the patient 101 is then sent from the blood withdrawal conduit 107 by the first pump 106 into the processing center 102 of the dual arm apheresis system 100 via the processing delivery conduit 111.

In the processing center 102 of the apheresis system 100, the desired portion of the whole blood is extracted. Apheresis systems can be configured to extract any component or components of whole blood, including but not limited to, platelets, leukocytes, erythrocytes, plasma, or combinations thereof. The extracted blood is then returned to the patient 101 via the delivery conduit 108.

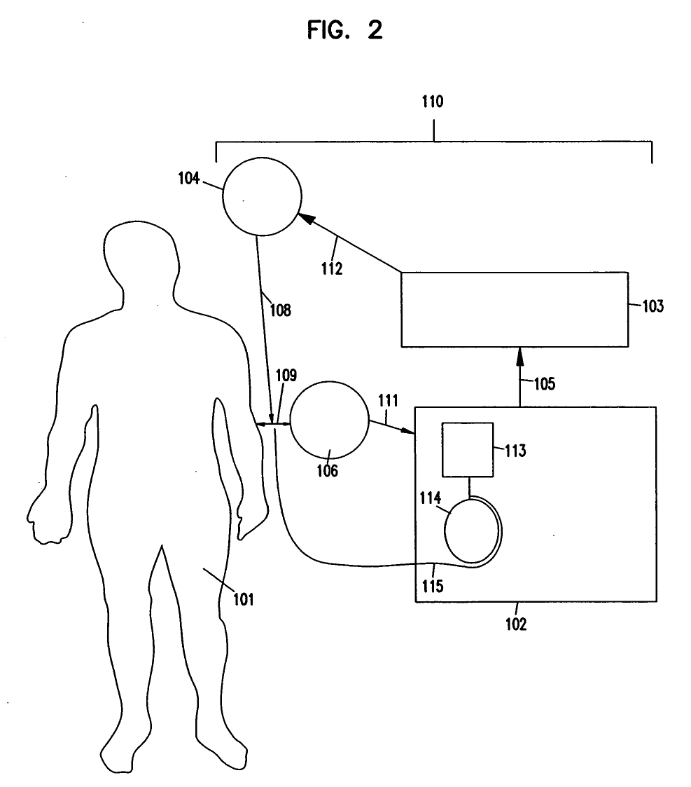

Apheresis systems are also available as single arm systems. A general schematic of the functioning of a single arm system is depicted in FIG. 2. The single arm system can be chosen from any of the currently available systems, such as Haemonetics Model V-50 (Haemonetics, Inc., Braintree Mass.) or the Baxter or Cobe models given above when configured for single arm apheresis, or any single arm apheresis system developed in the future. The whole blood is first withdrawn from the patient 101 by withdrawal/delivery conduit 109 by use of a first pump 106. The withdrawal/delivery conduit 109 is configured so that it can be used to withdraw whole blood from the patient 101 or deliver extracted blood to the patient 101, but cannot accomplish both simultaneously. An anticoagulant, contained in a first compartment 113, is delivered to the withdrawal/delivery conduit 109 by an anticoagulant pump 114 via an anticoagulant delivery conduit 115.

The whole blood is then sent to the processing center 102 via the processing delivery conduit 111. In the processing center 102 of the single arm apheresis system 110, the desired portion of the whole blood is extracted. The single arm apheresis system can be configured to extract any constituent component of whole blood, including but not limited to platelets, leukocytes, erythrocytes, plasma, or combinations thereof.

The extracted blood is then sent via a reservoir delivery conduit 105 to the reservoir 103. The extracted blood is contained in the reservoir 103 until the desired amount of blood component has been extracted from the whole blood. After the requisite amount of whole blood has been extracted, the extracted blood is then sent via the pump delivery conduit 112 to the second pump 104. The second pump 104 then pumps the extracted blood via the delivery conduit 108 into the withdrawal/delivery conduit 109. At this point, the withdrawal/delivery conduit 109 has been configured to allow the extracted blood to be delivered back into the patient 101.

Devices of the invention comprise a device for apheresis procedures that further comprises an antidote delivery system coupled to an anticoagulant delivery system. The delivery system can be volumetrically pumped or volumetrically metered, and is coupled to the blood delivery system. The antidote and anticoagulant delivery systems may be coupled mechanically, hydraulically, or electronically. Alternatively, the antidote delivery system may be coupled to the anticoagulant by virtue of the preparation of the solutions. The delivery systems may be peristaltic, piston, pneumatic, hydraulic, or other pumps known to those of skill in the art, and disclosed in the patents previously cited. A conduit, generally plastic tubing, extends from a reservoir of the antidote through a metering delivery device and terminates in conjunction with the component of the device of the invention that reinfuses the extracted blood into the donor. The antidote delivery system is correlated to the anticoagulant delivery system as discussed above in relation to methods of the invention and can be adjusted based on symptoms of the donor. The timing of administration of the antidote is adjusted so that ionic agent is not administered when the apheresis machine is not operating, and may be stopped at points prior to completion of the apheresis duration.

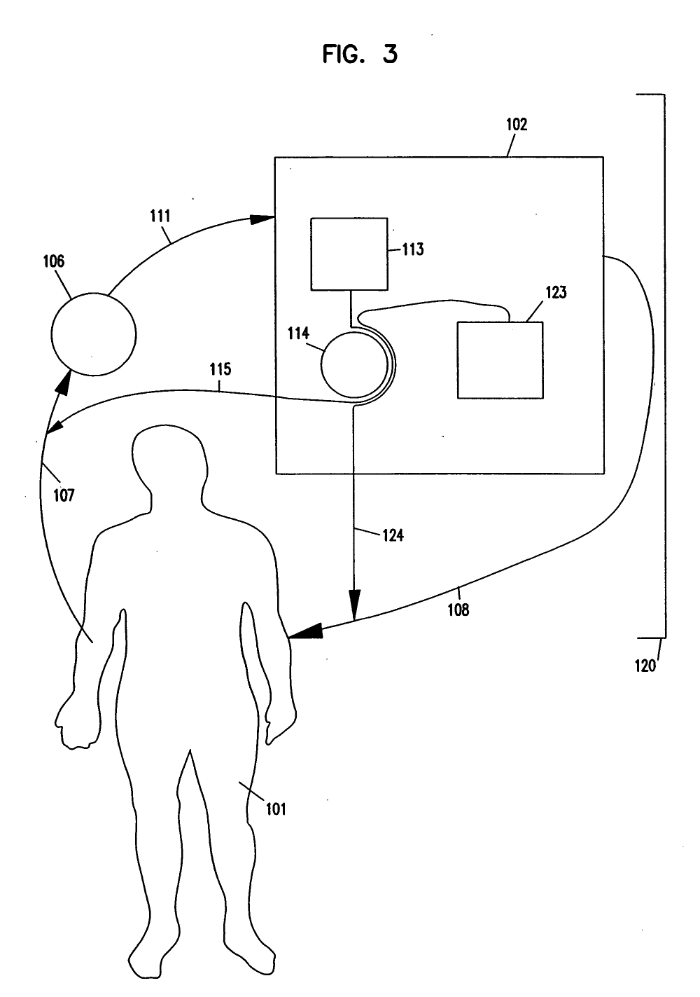

Devices of the invention can be constructed from standard dual or single arm apheresis machines already known in the art. Alternatively, devices of the invention can be constructed without the use of apheresis machines previously known in the art One embodiment of a device of the invention 120 incorporates a dual arm system; such as that described in reference to FIG. 1, and depicted in FIG. 3. Elements that are contained in the dual arm apheresis system 100 of FIG. 1 are numbered similarly, and are not explained again, except where they are modified or are important to the explanation of the device of the invention.

The device of the invention 120 comprises an antidote compartment 123. The antidote compartment 123 is preferably made and configured so it is easily incorporated into dual arm apheresis systems such as that depicted in FIG. 1. In one embodiment, the antidote compartment 123 can be similar to the anticoagulant compartment 113. The antidote delivery conduit 124 is attached to the antidote compartment 123 so that the antidote contained therein can be pumped via the anticoagulant pump 114, which is simultaneously pumping anticoagulant from the anticoagulant compartment 113. The antidote delivery conduit 124 is configured to deliver the antidote into the delivery conduit 108 so that it is mixed with the extracted blood before it is delivered to the patient 101.

The concentration of the antidote contained in the antidote compartment 123 is related to the concentration of the anticoagulant solution contained in the anticoagulant compartment 113. In this embodiment, the solutions will be pumped at the same rate because the same pump, the anticoagulant pump 114, is pumping the two solutions. Therefore, the two solutions are prepared so that the concentrations thereof result in the desired anticoagulant/antidote concentration ratio.

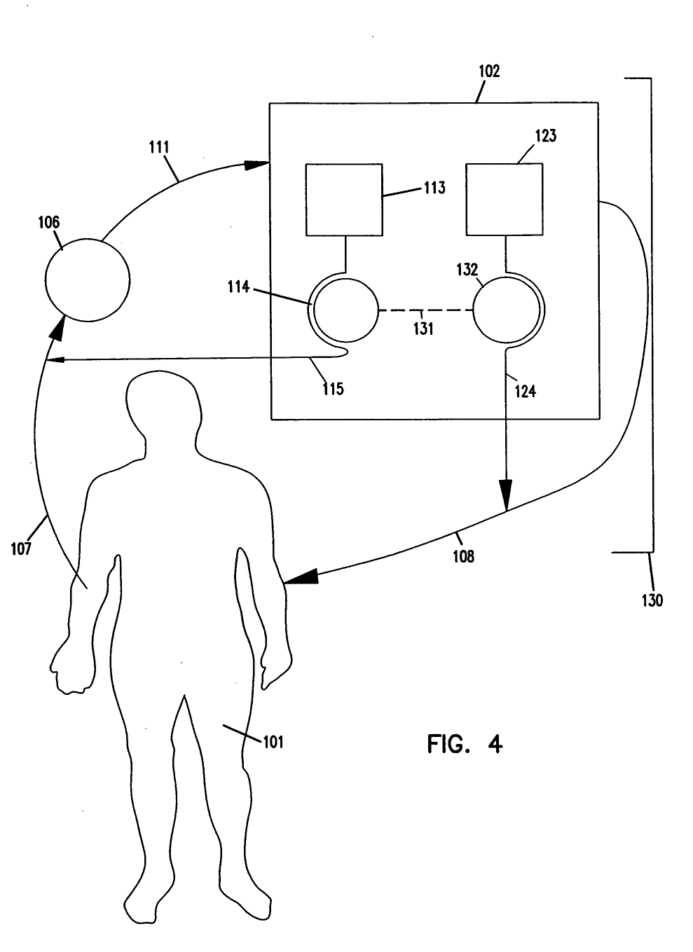

Another embodiment of a device of the invention 130 also incorporates a dual arm apheresis system, such as that described in reference to FIG. 1 and depicted in FIG. 4. Elements that are contained in the dual arm apheresis system of FIG. 1 are similarly numbered, and are not explained again, except where they are modified or are important to the explanation of the device of the invention.

The device of the invention 130 comprises an antidote compartment 123. The antidote compartment 123 is preferably constructed and configured so that it can be easily incorporated into dual arm apheresis systems such as that depicted in FIG. 1. The antidote delivery conduit 124 is attached to the antidote compartment 123 so that the antidote contained therein can be pumped via the antidote pump 132. In this embodiment, antidote pump 132 is coupled with the anticoagulant pump 114 via the pump coupling 131. The pump coupling 131 can be mechanical, hydraulic, or electronic. The antidote delivery conduit 124 is configured to deliver the antidote to the delivery conduit 108 so that it is mixed with the extracted blood before it is delivered to the patient 101.

In this embodiment, the concentration of antidote in antidote compartment 123 is not necessarily dependent on the concentration of the anticoagulant solution. An overall ratio of anticoagulant/antidote must still be maintained, but the necessary ratio can be obtained either by modifying the anticoagulant and antidote pumping rates or by correlating the concentrations of the anticoagulant and antidote. Therefore, this embodiment of the invention can offer more flexibility in anticoagulant and antidote preparation.

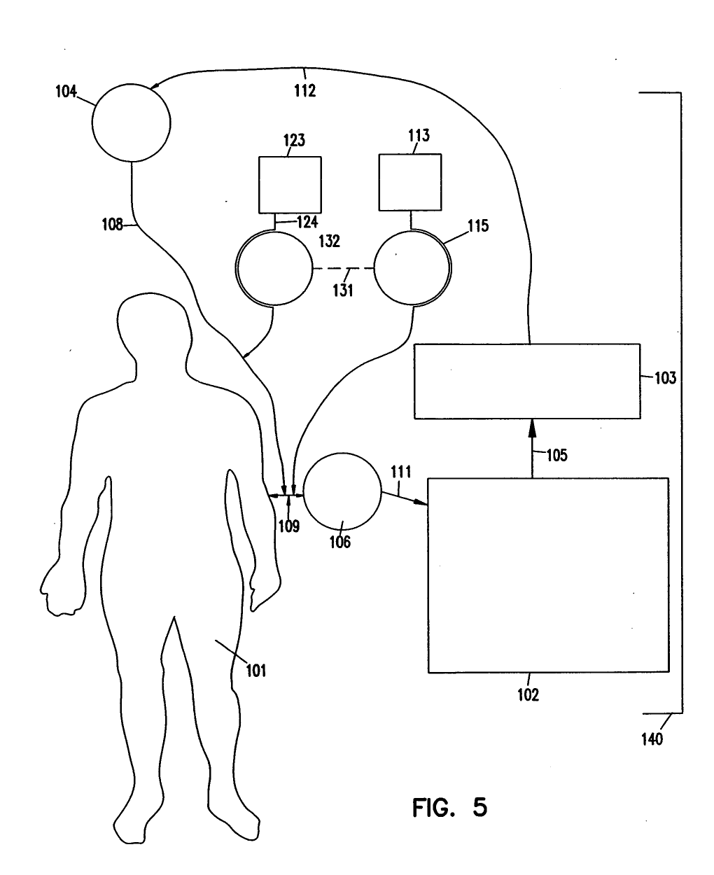

A further embodiment of a device of the invention 140 incorporates a single arm apheresis system such as that described in reference to FIG. 2 and depicted in FIG. 5. Elements that are contained in the single arm apheresis system of FIG. 2 are similarly numbered, and are not explained again, except where they are modified or are important to the explanation of the device of the invention 140.

The device of the invention 140 includes an antidote compartment 123. The antidote compartment 123 is constructed and configured so that it can be easily incorporated into single arm apheresis systems, such as that depicted in FIG. 2. The antidote delivery conduit 124 is attached to the antidote compartment 123 so that the antidote contained therein can be pumped via the antidote pump 132. The antidote pump 132 is coupled with the anticoagulant pump 114 via the pump coupling 131. The pump coupling 131 can be mechanical, hydraulic, or electronic. The antidote delivery conduit 124 is configured to deliver the antidote to the withdrawal/delivery conduit 109 so that it is mixed with the extracted blood before it is delivered to the patient 101.

In this embodiment of the invention, the concentration of the antidote in the antidote compartment 123 is not necessarily dependent on the anticoagulant solution. A specific ratio of anticoagulant/antidote must still be maintained, but the necessary ratio can be obtained either by modifying the anticoagulant and antidote pumping rates, or by correlating the concentrations of the anticoagulant and the antidote. Therefore, this embodiment of the invention can offer more flexibility in anticoagulant and antidote preparation.

In yet another embodiment of a device of the invention, a dialysis system is incorporated. In this setting, citrate is added to the blood prior to entering the dialysis device, and the ionic agents are added to the blood after completion of the dialysis, but before return of blood to the patient. The device may account for loss or addition of citrate across the dialysate membrane with the dialysate fluids.

Procedures for Using the Devices of the Invention

In operation, the tubings and associated fluid pathways of the device of the invention are filled with a priming solution of isotonic saline or isotonic saline with anticoagulant up to and including the tubing from the return line back to the antidote reservoir. It is important that the antidote delivery conduit be filled initially with the prime solution in order to permit anticoagulant rich fluids to be infused to the donor at the beginning of the apheresis procedure to assure adequate anticoagulation.

When apheresis begins, the general process withdraws blood from the donor and begins returning prime solution or blood with excess anticoagulant back to the donor. The antidote pump is delivering prime solution to the delivery conduit until that prime solution is replaced by antidote solution. The elapsed time between delivery of the first anticoagulant solution and the first antidote solution is between about one and ten minutes, preferably about six minutes.

Alternately, the tubings are all filled with their respective solutions and the antidote delivery system is started after the lapse of some time or volume of fluid delivered, about six minutes or about 100 ml of blood withdrawn may be typical.

### WORKING EXAMPLES

The following examples are provided as a non-limiting illustration of the invention.

### Example 1

Donors. All subjects were healthy allogeneic donors for lymphocyte or cytokine stimulated PBSC large volume leukapheresis procedures (LVL) who gave informed consent for apheresis and laboratory analysis on approved institutional protocols. Subjects in this study had normal hepatic and renal function tests, adequate peripheral venous access for a dual arm procedure without the use of a central apheresis catheter, were at least 18 years of age and weighed greater than 50 kg. For PBSC collections, subjects received 10 μg/kg daily for 6 days of subcutaneous granulocyte colony stimulating factor (GCSF) with LVL performed on the morning of day 5 and 6. Lymphocyte collections were performed prior to the first day of administration of GCSF. The estimated donor blood volume was calculated from the donor gender, height and weight.

Apheresis Procedures. LVL were performed using the small volume collection chamber on a CS-3000 cell separator (Baxter, Deerfield Ill.) with a maximum whole blood processing rate of 85 ml/min. The anticoagulant solution for all procedures was ACD-A (Baxter Healthcare, Fenwal Division, Deerfield Ill.) containing dextrose and 21.4 mg/ml of citrate as citric acid and trisodium citrate. Whole blood to ACD-A ratios of 12:1 and 13:1 (WB:AC) were employed to maintain product viability and adequate whole blood processing rates and to reduce the rate of citrate anticoagulant returned to the donor and thereby minimize donor symptoms. Controlled citrate infusions were achieved by maintaining a constant whole blood processing rate and a constant WB:AC ratio. Laboratory samples were obtained from a sterile-docked port inserted on the draw line 6 inches proximal to the infusion of ACD-A. Calcium infusions were administered in the return line through a standard port just proximal to the donor. Five ml of ACD-A was added to the product immediately after LVL, and autologous plasma and ACD-A added immediately after removal of the product from the apheresis device to achieve a final ACD-A concentration of 8% and a product volume of 300 ml.

Study Groups. LVL was performed at constant citrate infusion rates either with or without administration of intravenous prophylactic calcium solution infusions. Group A consisted of first-time donors who underwent LVL of 12-15 L processed at standard citrate infusion rates between 1.0 and 1.6 mg/kg/min without administration of prophylactic calcium infusions. Group B consisted of first-time and repeat donors who underwent LVL of 15-25 L processed at higher citrate infusion rates of 1.6-2.2 mg/kg/min with administration of prophylactic calcium starting at the beginning of the procedure.

Studies were also performed in 15 donors to evaluate changes 24 hours after LVL, and in 7 donors to determine the dose response of intravenous magnesium infusions. Clinical features of additional procedures performed with prophylactic calcium and magnesium solutions were analyzed along with data from these laboratory studies to develop a standard protocol for management of citrate related symptoms during LVL.

Laboratory Measurements. Blood samples were obtained at 0, 30, 60, 120, 180 minutes; hourly thereafter during LVL; at the end of LVL; 30 and 90 minutes after LVL; and at the development of donor symptoms≧level 2. Sera from blood samples was collected anaerobically and sent for immediate analysis of ionized calcium, magnesium and pH with an AVL 988-4 (AVL Scientific, Roswell Ga.). Citrate levels were measured enzymatically using a COBAS FARA machine (Roche Diagnostics Systems Inc., Montclair, N.J.). Total calcium and magnesium, sodium, potassium, bicarbonate, glucose, and other blood chemistries were measured by standard techniques in routine clinical use. Plasma samples for intact parathyroid hormone (PTH) were analyzed using an IMMULITE® Automated Assay System (Diagnostics Products Corporation, Los Angeles, Calif.). Spot urine samples were analyzed before and after LVL for total calcium and magnesium, citrate, creatinine and pH.

Intravenous Infusions. Equimolar calcium gluconate and calcium chloride were prepared by the pharmacy from 10% solutions to contain a final concentration of 2 mg calcium ion per ml. (calcium chloride (Fujisawa USA, Deerfield Ill.) four 10 ml vials, 1092 mg elemental calcium, final volume 546 ml; calcium gluconate (Fujisawa USA, Deerfield Ill.) twelve 10 ml vials, 1116 mg elemental calcium, final volume 558 ml) The measured osmolality for ACD-A was 394 mosm/kg, for calcium chloride 391 mosm/kg in normal saline and 268 mosm/kg in half normal saline, and for calcium gluconate 310 mosm/kg in normal saline and 201 mosm/kg in half normal saline. Magnesium infusions were prepared by adding 3 ml (24 meq) of 50% magnesium sulfate (American Pharmaceutical Partners Los Angeles Calif.) to normal saline in a final volume of 98.6 ml, providing 3 mg of magnesium ion per ml of solution. Cost estimates for preparation of solutions were based on government costs for calcium chloride of $0.38 per 10 ml vial and calcium gluconate of $0.97 per 50 ml vial, with 5 minutes technical preparation time and 5 minutes pharmacist time.

Calcium was administered at 0.5 mg calcium ion per ml of ACD-A (1 mmole calcium per 10 mmoles citrate). Donors in Group A received calcium beginning at the onset of symptoms≧level 2. Donors in Group B received calcium prophylactically beginning 5 minutes after initiation of LVL. Calcium infusions were stopped immediately if LVL was halted or 5 minutes prior to the completion of LVL. Magnesium was also administered to Group B at 0.15 mg of magnesium ion per ml of ACD-A (0.5 mmole magnesium per 10 mmoles citrate). Procedures were conducted at constant WB:AC ratios. Doses of calcium (and magnesium when utilized) solutions were therefore administered according to the whole blood processing rate. At a WB:AC ratio of 13:1, the rate of administration (in ml/hr) of the 2 mg/ml calcium solutions was 1.07 times the whole blood processing rate (in ml/min). When magnesium was administered, the rate of the 3 mg/ml magnesium solution was 20% of the calcium infusion rate.

Donor Symptom Assessment and Management. Donor symptoms were assessed by experienced apheresis nurses as “0” none, “1” barely noticeable, “2” irritating, “3” uncomfortable, and “4” unbearable. For symptoms≧2, intravenous calcium was initiated in Group A. For donor symptoms≧3, the whole blood processing rate was decreased by 20%. For symptoms≧4, the procedure was stopped.

Statistical Analysis. The proportion of donors with symptoms at each citrate infusion rate was calculated using the two-tailed Kruskal-Wallis test for ordered column contingency methods. Symptoms between men and women donors were compared with Thomas's exact test for stratified two by two contingency tables at four citrate infusion rates in group A. Significance tests on paired samples from donors on the day before and day after LVL were performed with a paired two tailed T-Test, while samples between groups were conducted with a two-tailed, non-paired T-test. Error bars on graphs are the standard error of the mean.

Results

Donor Responses. Donor demographics and symptom responses are shown in Table 1 below.

TABLE 1

| Citrate | AC/BV | | Sex | Wt | WBFR | Time | Symptom Scores | | |
|---|---|---|---|---|---|---|---|---|---|
| (mg/kg/min) | (ml/L/min) | n | M/F | (kg) | (ml/min) | (min) | “0” | “≧1” | “≧2” |
| Group A | | | | | | | | | |
| 1.0 | 0.81 | 6 | 3/3 | 82 | 55 | 257 | 5/6 | 1/6 | 0/6 |
| 1.2 | 0.9 | 6 | 3/3 | 77 | 59 | 245 | 3/6 | 3/6 | 1/6 |
| 1.4 | 0.99 | 6 | 4/2 | 69 | 62 | 224 | 2/6 | 4/6 | 2/6 |
| 1.6 | 1.2 | 6 | 3/3 | 70 | 71 | 205 | 1/6 | 5/6 | 2/6 |
| | | | | | | | 11/24 | 13/24 | 5/24 |
| Group B | | | | | | | | | |
| 1.6 | 1.19 | 10 | 4/6 | 80 | 80 | 200 | 9/10 | 1/10 | 0/10 |
| 1.8 | 1.36 | 6 | 2/4 | 68 | 78 | 205 | 5/6 | 1/6 | 0/6 |
| 2.0 | 1.41 | 5 | 2/3 | 60 | 77 | 204 | 4/5 | 1/5 | 0/5 |
| 2.2 | 1.44 | 4 | 2/2 | 56 | 84 | 194 | 3/4 | 1/4 | 0/5 |
| | | | | | | | 21/25 | 3/25 | 0/25 |

The percentage of men and women in each group was similar. Procedures at higher citrate infusion rates tended to be shorter due to faster blood processing rates. In procedures performed without calcium (Group A), the percentage of donors with grade 1 and 2 symptoms increased with increasing citrate infusion rates. Only one donor had symptoms≧1, and no donors had symptoms≧2 at the lowest citrate infusion rate of 1.0 mg/kg/min. Five of six donors had symptoms≧1 at a citrate infusion rate of 1.6 mg/kg/min, and two of six donors experienced symptoms≧2 at each citrate infusion rate of 1.4 and 1.6 mg/kg/min. The increase in level 1 symptoms from 1.0 to 1.6 mg/kg/min was statistically significant (p=0.02) by the ordered column contingency test, while the change in level 2 symptoms did not reach statistical significance (p=0.12). Level 1 symptoms were reported by 8/11 women (73%) and 5/13 men, (38%), while level 2 symptoms were reported by 4/11 women (36%) and 1/13 men (8%). The difference in the incidence of level 1 (p=0.13) and level 2 (p=0.17) symptoms was not statistically significant in men and women. In all cases, the development of level 2 symptoms was preceded by level 1 symptoms.

No donors in Group A or B progressed to symptoms of level 3 or 4. One donor rapidly developed level 4 symptoms during her second daily LVL performed at a citrate infusion rate of 1.4 mg/kg/min without prophylactic calcium. Level 2 symptoms progressed rapidly despite initiation of treatment with intravenous calcium gluconate. Her symptoms resolved twenty to thirty minutes after discontinuation of blood processing, and she subsequently received 3 additional LVL performed with prophylactic calcium at citrate infusion rates of 1.6 mg/kg/min without symptoms.

There were no symptoms≧2 in the 24 procedures performed with prophylactic intravenous calcium at citrate infusion rates up to 2.2 mg/kg/min in group B.

Laboratory Values. Average blood citrate levels increased progressively with increasing citrate infusion rates during LVL. There was no evidence of stabilization of average blood citrate levels over the course of LVL at citrate infusion rates greater than 1.2 mg/kg/min. Notably, at the 90 minute time point when plateletpheresis procedures are usually concluded, blood citrate levels were still clearly increasing at all citrate infusion rates. Blood citrate levels varied significantly between donors during LVL performed at the same citrate infusion rate, but were much more consistent in the same donor during repeat LVL performed at same citrate infusion rates. Similar inter-donor variability was seen at other citrate infusion rates, and inter-donor responses were more variable than intra-donor responses.

These marked increases in blood citrate levels were accompanied by profound decreases in ionized calcium in LVL performed at standard citrate infusion rates without prophylactic calcium administration. Progressively more marked decreases in ionized calcium were observed in these donors at increasing citrate infusion rates, with nadir values up to 35% below baseline and ionized calcium levels as low as 0.84 mmoles/L. There were no level 2 symptoms in group A when ionized calcium levels were greater than 1.00 mmoles/L. Although not all donors reported symptoms at lower ionized calcium levels, ionized calcium levels tended to be lower in donors with symptoms compared to those without symptoms.

The decreases in ionized calcium levels and associated symptoms were significantly attenuated when prophylactic calcium was administered, despite much higher citrate infusion rates, higher blood citrate levels and much larger processed blood volumes (18 L average versus 13 L p<0.000005) compared to those without calcium. No donor given prophylactic calcium had symptoms≧level 2 during the procedures, and nadir ionized calcium levels were maintained greater than 1.00 mmoles/L except at the highest blood citrate levels (FIG. 3a).

Barely noticeable level 1 paresthesias occurred in 3 donors given calcium gluconate and in 1 donor given calcium chloride. All four donors with symptoms were female donors with low blood CD34 counts undergoing a second or third consecutive LVL of more than 20 L to meet a target cell dose. Nadir ionized calcium levels (1.03 vs 1.13, p=0.004) as well as nadir ionized magnesium levels (0.22 vs 0.30 mmoles/L, p=0.002) were significantly decreased in these donors compared to those without symptoms during prophylactic calcium infusions.

There was no difference in blood ionized or total calcium levels as a function of blood citrate concentration in the donors who received calcium gluconate compared to calcium chloride.

Decreased ionized magnesium levels were also observed in association with increased blood citrate levels rates during LVL. The relationship between pre-apheresis ionized cation levels and low baseline citrate levels was similar to that observed during apheresis when blood citrate was markedly elevated, and the calculated intercept at zero citrate concentration of ionized levels versus blood citrate of both magnesium (0.57 mmoles/L) and ionized calcium (0.129 mmoles/L) was in the normal range. The most profound decreases in ionized magnesium occurred in the donors receiving prophylactic calcium administration at high citrate infusion rates. Ionized magnesium levels were significantly decreased in 4 donors who developed barely noticeable paresthesias during prophylactic calcium administration, however ionized calcium levels were also decreased in these donors. Percent decreases in ionized magnesium as large as 50% associated with nadir absolute values below 0.20 mmoles/L were observed at the conclusion of longer procedures.

Changes in blood ionized calcium and magnesium were strongly related to blood citrate levels. The blood ionized calcium, expressed as the fraction of total calcium ([ionized calcium]/[total calcium]), was highly correlated with blood citrate concentration, and was indistinguishable in procedures performed with or without calcium. In LVL performed without prophylactic calcium, total calcium levels remained relatively unchanged, however ionized calcium levels decreased progressively and steadily as the fraction of ionized calcium decreased with increasing blood citrate levels. In LVL performed with administration of prophylactic calcium, total calcium levels increased and ionized calcium levels decreased more gradually as blood citrate levels increased. In these procedures, the fraction of total calcium present as ionized calcium was unchanged in relationship to blood citrate, however the decrease in ionized calcium levels was attenuated due to the increased total calcium concentration. Thus, over the course of LVL, ionized calcium levels fell progressively and symptoms increased in donors who did not receive prophylactic calcium, while these changes were clinically and significantly minimized in donors who received prophylactic calcium.

In contrast, the increased blood parathyroid (PTH) levels did not have a constant relationship to changes in blood-ionized calcium over the course of LVL. PTH levels were highest at 30 minutes in all procedures, but then fell despite continued decreases in ionized calcium. Peak levels were 450% above baseline in donors who did not receive prophylactic calcium, and 70 to 160% above baseline in the donors who did. In group A; the peak-, mid-, end-, and post-apheresis PTH levels were similar in all citrate infusion rates despite marked differences in ionized calcium. During LVL, PTH levels remained increased compared to baseline, but were decreased compared to the 30 minute levels despite continued progressive decreases in ionized calcium. In group B, the peak PTH levels were also highest at 30 minutes, but then decreased during the remainder of apheresis and were below baseline by the end of the procedure.

Urinary excretion of citrate increased markedly in samples measured immediately after LVL compared to those obtained before apheresis. Table 2 below shows results of spot urine chemistry tests. The results are given as a ratio of the concentrations in samples taken pre-LVL and post-LVL.

TABLE 2

| | Citrate | Calcium | Magnesium |
|---|---|---|---|
| Citrate InfusionRate | Post/Pre LVL | Post/Pre LVL | Post/Pre LVL |
| (mg/kg/min) | ratio | ratio | ratio |
| Group A | | | |
| 1.0 | 13 (7) | 2.0 (0.8) | 1.8 (1.0) |
| 1.2 | 27 (9) | 3.0 (3.7) | 2.9 (2.3) |
| 1.4 | 42 (26) | 3.0 (0.7) | 2.0 (0.9) |
| 1.6 | 25 (17) | 1.7 (1.4) | 2.5 (0.9) |
| Group B | | | |
| 1.6 | 30 (40) | 12.5 (16) | 5.4 (3.3) |
| 1.8 | 30 (18) | 20 (23) | 7.0 (7.3) |
| 2.0 | 31 (18) | 7.7 (4.2) | 2.7 (1.1) |
| 2.2 | 30 (3) | 12.3 (5.8) | 5.7 (0.4) |

Despite increased blood PTH and decreased blood ionized calcium and magnesium levels, urine chemistries demonstrated marked increases in calcium excretion after LVL, as well as marked increases in urinary magnesium excretion. The excretion of calcium and magnesium was further increased in donors who received prophylactic intravenous calcium infusions at high citrate infusion rates. Blood levels of potassium and phosphate decreased significantly during LVL, but returned toward normal limits at 90 minutes after completion, while bicarbonate and pH increased during LVL and after LVL. The decreases in potassium were attenuated during the procedures performed with prophylactic calcium, and were significantly lower 90 minutes after LVL in procedures performed without prophylactic calcium compared to those performed with prophylactic calcium.

Laboratory testing was performed on morning after LVL (day 2) in 15 donors who returned for a repeat procedure and compared with samples obtained before LVL (day 1) to evaluate possible longer lasting changes in blood and urine chemistries. Day 2 PTH levels increased by 55% (p=0.02) compared to day 1 in the six donors who did not receive prophylactic calcium during their first procedure. In contrast, PTH levels in the nine donors who received prophylactic calcium were not significantly changed on day 2 compared to day 1 (28% decrease, p=0.15). Total blood calcium levels exhibited similar changes in LVL performed without calcium (−4.4% p=0.032) and with calcium (−3.4% p=0.062) on day 2 compared to day 1. Ionized calcium levels were significantly decreased in LVL performed without calcium (3.5%, p=0.02) and in those performed with calcium (3.0%, p=0.03) on day 2 compared to day 1. Significant decreases in blood ionized (12.6%, p=0.001) and total (14.4%, p=0.004) magnesium levels were observed on day 2 in the procedures performed at high citrate infusion rates with prophylactic calcium. Day 2 changes were not significant compared to day 1 in ionized (−2.1%, p=0.46) and total (−0.7%, p=0.70) magnesium in LVL performed at standard citrate infusion rates without calcium prophylaxis. Changes in blood measurements were accompanied by non-significant decreases in excretion of urinary calcium (19% group A p=0.47, 12% group B p=0.61) and magnesium (27% group A p=0.26, 36% group B p=0.06). The day 2 changes were significantly different between donors that received high citrate infusions with prophylactic calcium compared to those that received standard citrate infusion rates for PTH (p=0.02), ionized (p=0.003) and total magnesium (p=0.004), but not for other measured parameters.

### Example 2

Prophylactic intravenous calcium was also administered in an additional 240 LVL performed in adults at citrate infusion rates between 1 and 2.6 mg/kg/min with an average of 15 L processed. Intravenous magnesium was prophylactically administered in addition to calcium in 17 of these procedures, in which the average citrate infusion rate was 1.92 mg/kg/min and the average blood volume processed was 21 L. Mild paresthesias were observed in 4 of these 17 donors. Calcium without magnesium was administered to the remaining 223 procedures, which were performed at an average citrate infusion rate of 1.63 mg/kg/min (including 40>2.0 mg/kg/min and 69>1.8 mg/kg/min), with an average volume processed of 14.7 L. Mild symptoms occurred in 39 donors, 13 at citrate infusion rates greater than, and 26 at citrate infusion rates less than 1.8 mg/kg/min. The whole blood processing rate was decreased in two donors to control persistent symptoms. There were two episodes of dizziness, no symptoms>level 2, and no vaso-vagal episodes. No complications have been observed with the apheresis product or coagulation in the apheresis device.

### Example 3

Based on the above examples, the following protocol is recommended. Prophylactic calcium is administered to all donors undergoing LVL at citrate infusion rates≧1.2 mg/kg/min. Prophylactic calcium is also administered to all donors who experienced symptoms during prior LVL at lower citrate infusion rates. Calcium chloride is administered as 2 mg/ml in half normal saline, because of its lower cost of preparation ($6.77 per 500 ml bag of calcium chloride compared to $8.34 for calcium gluconate). Calcium chloride solutions are administered at 0.5 mg per ml of ACD-A for citrate infusion rates <2.0 mg/kg/min and at 0.6 mg per ml of ACD-A for citrate infusion rates≧2.0 mg/kg/min. If donors develop level 1 paresthesias, the calcium infusion is increased gradually up to 0.65 mg per ml of ACD-A. The whole blood processing rate is also decreased by 10-20% for persistent level 1 paresthesias, or for symptoms≧level 2. Donors undergoing LVL which process more than 4 donor blood volumes also receive prophylactic magnesium, 3 mg/ml in normal saline, at 20% of the calcium infusion rate. Magnesium solutions are also administered to donors undergoing repeat LVL if paresthesias developed during prior LVL.

The working examples given above clearly demonstrate the use of calcium solutions and their safety in LVL. Moreover, analysis of the changes in calcium levels over the time course of LVL revealed that marked decreases occurred during the time frame in which the platletpheresis procedures are normally done when utilizing these same citrate infusion rates. Follow up studies have also confirmed the existence of these laboratory changes in platelet donation as well as the occurrence of symptoms that are uncomfortable to the donors and which can significantly limit the dose of platelets obtained from the procedure. Therefore the methods and devices of the invention have applicability to platelet procedures as well as LVL. In one embodiment of the invention, devices and methods thereof can be used in any apheresis procedure to counteract toxicity in a donor from the return to the donor of an anticoagulant that must be administered to prevent clotting of the product or blood in the apheresis device.

The above specification, examples and data provide a complete description of the manufacture and use of the composition of the invention. Since many embodiments of the invention can be made without departing from the spirit and scope of the invention, the invention resides in the claims hereinafter appended.

1-47. (canceled)

48. An apheresis device for extraction of one or more constituent components of whole blood from a mammal, the device comprising:
a) a blood withdrawal system for removing blood from the mammal and delivery of the blood to the apheresis device;
b) an anticoagulant delivery system connected to the blood withdrawal system for delivery of an anticoagulant to the blood;
c) a processing center, connected to the blood withdrawal system, wherein one or more constituent components are extracted from the blood;
d) a delivery conduit connecting the processing center to the mammal, for reinfusing the blood with one or more constituent components removed; and
e) an antidote delivery system, connected to the delivery conduit near the point of reinfusion, for delivery to the blood of an antidote to the anticoagulant; and
wherein the amount of anticoagulant delivered by the anticoagulant delivery system is coupled to the amount of antidote delivered by the antidote delivery system.

49. The apheresis device of claim 48, wherein a constituent component extracted comprises stem cells.

50. The apheresis device of claim 48, wherein the constituent components are leukocytes, erythrocytes, platelets, plasma, hematopoietic transplantable cells, other blood cells, or a combination thereof.

51. The apheresis device of claim 48, wherein said mammal is a human.

52. The apheresis device of claim 48, wherein said anticoagulant is a citrate compound, heparin, EDTA, or a combination thereof.

53. The apheresis device of claim 53, wherein said citrate compound comprises dextrose, citric acid, trisodium citrate, or combinations thereof.

54. The apheresis device of claim 48, wherein said antidote comprises calcium, magnesium, potassium, or combinations thereof.

55. The apheresis device of claim 48, wherein said antidote is calcium chloride, calcium gluconate, or mixtures thereof.

56. The apheresis device of claim 48, wherein said antidote comprises magnesium sulfate.

57. The apheresis device of claim 48, wherein the anticoagulant is ACD-A and the antidote comprises a calcium compound wherein the amount of anticoagulant delivered is coupled to the amount of antidote by the applying the ratio of 0.2-2.0 mg calcium ion per mL of ACD-A.

58. The apheresis device of claim 48, wherein the anticoagulant is ACD-A and the antidote is a calcium compound wherein the amount of anticoagulant delivered is coupled to the amount of antidote by the applying the ratio of 0.6-0.8 mg calcium ion per mL of ACD-A.

59. The apheresis device of claim 48, wherein the amount of anticoagulant delivered by the anticoagulant delivery system is coupled to the amount of antidote delivered by the antidote delivery system by applying the ratio of mmoles of antidote to mmoles of citrate anticoagulant in a range from 0.01-1.

60. The apheresis device of claim 48, wherein the amount of anticoagulant delivered by the anticoagulant delivery system is coupled to the amount of antidote delivered by the antidote delivery system by applying the ratio of mmoles of antidote to mmoles of citrate anticoagulant in a range from 0.01-0.15.

61. The apheresis device of claim 48, wherein the amount of antidote delivered is related to rate of delivery, and the rate of delivery of the antidote is adjustable within a range of values, by an operator of the apheresis device based on donor symptoms and vital signs.

62. The apheresis device of claim 48, wherein the anticoagulant system comprises an anticoagulant-carrying conduit operably connected to a first pump; and wherein the antidote delivery system comprises an antidote-carrying conduit operably connected a second pump; wherein the delivery rates of the first pump and second pump are coupled, thereby coupling the amount of antidote delivered to the amount of anticoagulant delivered.

63. The apheresis device of claim 62, wherein the anticoagulant comprises a citrate compound and the amount of citrate in said anticoagulant administered to the donor ranges from about 0.4 mg/kg body weight of said mammal/minute to 6 mg/kg body weight of said mammal/minute.

64. The apheresis device of claim 63, wherein the antidote comprises calcium ion administered at 0.01 to 0.3 mmoles calcium per 1 mmol citrate.

65. The apheresis device of claim 64, wherein the antidote delivery system and the anticoagulant delivery system are coupled by computer circuitry between a pump for delivery of said anticoagulant and a pump for delivery of said antidote.

66. The apheresis device of claim 48, wherein the anticoagulant system comprises an anticoagulant-carrying conduit operably connected to a pump; and wherein the antidote delivery system comprises an antidote-carrying conduit operably connected to the pump of the anticoagulant system; wherein rate of delivery of the antidote and rate of delivery of anticoagulant are coupled by utilizing a single pump for delivery of both the anticoagulant and the antidote, thereby coupling the amount of antidote delivered to the amount of anticoagulant delivered.

67. The apheresis device of claim 66, wherein said coupling of the antidote delivery system and the anticoagulant delivery system is accomplished by preparing solution of anticoagulant and a solution of antidote so that the amount of antidote delivered is correlated to the amount of anticoagulant delivered.

68. The apheresis device of claim 67, wherein the anticoagulant comprises citrate and the antidote comprises calcium ion administered at 0.01 to 0.3 mmoles calcium per 1 mmol citrate.

69. The apheresis device of claim 48, wherein the blood withdrawal system comprises a blood withdrawal conduit operably connected to a first pump; and wherein the anticoagulant system comprises an anticoagulant-carrying conduit operably connected to a second pump; and wherein the antidote delivery system comprises an antidote-carrying conduit operably connected to a third pump; and wherein the delivery rates of the first pump, second pump and third pump are coupled.

70. The apheresis device of claim 69, wherein the rate of blood withdrawal by the first pump, the rate of delivery of anticoagulant by the second pump and the rate of delivery of the antidote, are adjustable within a range of values, by an operator of the apheresis device based on donor symptoms and vital signs.

71. The apheresis device of claim 69, wherein the delivery rate of the antidote by the third pump is from about 0.001-5.0 times the delivery rate at which the blood is withdrawn from said mammal by the first pump.

72. The apheresis device of claim 69, wherein the delivery rate of the antidote by the third pump is from about 0.01-1 times the delivery rate at which the blood is withdrawn from said mammal by the first pump.

73. A method of using the apheresis device of claim 48, wherein continuous apheresis is performed on a blood volume corresponding to one or more total blood volumes of the individual donor.

74. The method of claim 73, wherein apheresis is performed for the collection of stem cells.

75. The method of using the apheresis device of claim 69, wherein the device is used for discontinuous apheresis.
